(19) United States
(12) Patent Application Publication
(10) Pub. No.: US 2013/0046121 A1
(43) Pub. Date: Feb. 21, 2013

(54) Processes and Systems for Recovery of Residual Halogenated Hydrocarbons in the Conversion of Natural Gas to Liquid Hydrocarbons

(71) Applicants: Sabah A. Kurukchi, Houston, TX (US); Yijun Liu, Houston, TX (US); Anand Moodley, Houston, TX (US)

(72) Inventors: Sabah A. Kurukchi, Houston, TX (US); Yijun Liu, Houston, TX (US); Anand Moodley, Houston, TX (US)

(73) Assignee: MARATHON GTF TECHNOLOGY, LTD., Houston, TX (US)

(21) Appl. No.: 13/212,291

(22) Filed: Aug. 18, 2011

(51) Int. Cl. C07C 17/38; B01J 8/00; C07C 5/00; F25J 3/08

(52) U.S. Cl. 570/241; 62/630; 422/187; 585/700

(57) ABSTRACT

Process and systems for converting lower molecular weight alkanes to higher molecular weight hydrocarbons that include recovery of residual halogenated hydrocarbons (e.g., CH₃Br) from higher molecular weight hydrocarbon products.

### BACKGROUND

The present invention relates generally to processes and systems for converting lower molecular weight alkanes to higher molecular weight hydrocarbons and, more particularly, in one or more embodiments, to processes for converting lower molecular weight alkanes that include recovery of halogenated hydrocarbons from higher molecular weight hydrocarbon products.

Natural gas, which is primarily composed of methane and other light alkanes, has been discovered in large quantities throughout the world. In the United States, the latest proved natural gas reserves are 6,731 billion standard cubic meters (238 trillion standard cubic feet) in 2010, which makes the United States a top-five country in natural gas abundance. Natural gas is generally a cleaner energy source than crude oil. It is normally heavy sulfur-free and contains none or a minimum amount of heavy metals and non-reacting heavy hydrocarbons. For a given amount of heat energy, burning natural gas produces about half as much carbon dioxide as coal.

However, the transportation, storage and distribution of natural gas in a gaseous form are much less favorable than those of crude oil making it more difficult to be a substitute as the predominant energy source. Converting natural gas to higher molecular weight hydrocarbons, which, due to their higher density and value, are able to be more economically transported, can significantly aid the development of natural gas reserves, particularly the stranded remote natural gas reserves.

One technique for converting natural gas to higher molecular weight hydrocarbons is a bromine-based process. In general, the bromine-based process may include several basic steps, as listed below.
(1) Bromination: Reacting bromine with lower molecular weight alkanes to produce alkyl bromides and hydrogen bromide (HBr).
(2) Alkyl Bromides Conversion: Reacting the alkyl bromides over a suitable catalyst under sufficient conditions to produce HBr, methane (C1), light end hydrocarbons (C2-C4) and heavy end hydrocarbons (C5+).
(3) HBr Recovery: Recovering HBr produced in both steps (1) and (2) by one of several processes, e.g., absorbing HBr and neutralizing the resulting hydrobromic acid with an aqueous solution of partially oxidized metal bromide salts (as metal oxides/oxy-bromides/bromides) to produce metal bromide salt and water in an aqueous solution; reacting HBr with metal oxide; or absorbing HBr into water using a packed tower or other contacting device.
(4) Bromine Regeneration: Reacting the bromide recovered in step (3) with oxygen or air to yield bromine and treating it sufficiently for recycle to step (1).
(5) Product Recovery: Fractionating by distillation and cryogenic distillation (demethanizer) the hydrocarbon mixtures contained in the effluent from step (2) and then separated from HBr in step (3) into methane, light end hydrocarbons, and heavy end hydrocarbons. The methane can be compressed for recycle to step (1). The light end hydrocarbons (C2-C4) may be, for example, salable as a product or cracked to produce light olefins. The heavy end hydrocarbons (C5+) may be used, for example, for further petrochemical or fuel processing.

In alkyl bromides conversion, the exothermic coupling reaction may be carried out in a fixed-bed, fluidized-bed or other suitable reactor in the presence of suitable catalysts under sufficient conditions (e.g., 150-600° C., 1-80 bar). The catalyst may have to undergo decoking periodically or continuously to maintain adequate performance. In some instances, a fluidized-bed reactor may be considered to be advantageous for the coupling reaction, particularly for commercial scale of operation, as it should allow for continuous removal of coke and regeneration of the spent catalyst without requiring daily shutdowns and expensive cyclic operation. However, the nature of the fluidized-bed reactor may make it difficult to achieve complete conversion of mono-bromomethane (CH₃Br), typically the primary reactant in the case of converting natural gas to liquid hydrocarbons. In some instances, wherein the catalyst deactivation rate is lowered by feeding none or a minimum amount of polybrominated alkanes to the alkyl bromides conversion step, the fixed-bed configuration may be preferred over the fluidized bed. In the latter case, the fixed-bed reactor is typically allowed to operate continuously over a period until the conversion of CH₃Br drops to a predetermined threshold (e.g., about 90%). Furthermore, CH₃Br conversion is also highly sensitive to the operating conditions of the reactor, e.g., reaction temperature, space velocity, time on stream, number of catalyst regeneration cycles, etc., which adds additional factors leading to an appreciable and fluctuating amount of unconverted CH₃Br leaving the reactor with HBr and higher molecular weight hydrocarbons. The presence of alkyl bromides in the product streams can limit the use or sale of the higher molecular weight hydrocarbons for further petrochemical or fuel processing.

The removal of halogenated hydrocarbons from product or emission streams has also been of a concern in other industries, such as the production of plastics and herbicides. There have been efforts to develop efficient processes for dehalogenation. Most of the previously proposed methods typically involve the use of chemical destruction through incineration, catalytic decomposition, or catalytic hydrogenation in presence of a suitable hydrogen source and/or oxygen source. In one example, butane is used as a hydrogen source to debrominate more than 98% of CH₃Br in the presence of oxygen over a noble metal-alumina catalyst at 500-550° C. and 1 atmosphere. Another example is high-temperature gas phase reductive dehalogenation of polyhalogenated hydrocarbons by direct reaction with molecular hydrogen over a 10% Ni on ZSM5 catalyst supported on alumina. In another example, metal oxide (e.g., MgO, ZrO₂, Al₂O₃, or zeolite) reacts with CH₃Br and water to yield HBr and methanol. Yet another example involves reacting halogenated hydrocarbons over a Pt on alumina catalyst in the presence of methanol or alkane solvents to yield dehalogenated hydrocarbons and acid halide. If a metal oxide catalyst such as MnO₂ is used instead of Pt/Al₂O₃, halogenated hydrocarbons can be completely destructed to carbon dioxide at 300-400° C.

The aforementioned methods for dehalogenation have some drawbacks in a bromine-based process for converting natural gas to higher molecular weight hydrocarbons. First, the introduction and/or production of oxygen-containing species such as air, alcohol, water, and carbon dioxide is typically not desirable for the removal of CH₃Br from the synthesis reactor effluent, as it would lead hydrocarbon loss to carbon dioxide and water and/or generate a highly corrosive aqueous/alcoholic HBr stream, thus complicating the process metallurgy. Second, a source of molecular hydrogen is not typically available as a byproduct of this process, requiring a thermal cracker or electronic cell to be built separately to produce H₂ on site. Third, selective debromination catalyst is necessitated as the olefinic and aromatic hydrocarbon products contained in the synthesis reactor effluent are prone to saturation in presence of active hydrogen donors. Such saturation would be counterproductive. Fourth, essentially all of catalytic dehalogenation methods mentioned above suffer from difficulties such as incomplete dehalogenation, catalyst deactivation, the need of catalyst regeneration and/or replacement, expensive cyclic operation, and limited process reliability. Furthermore, a catalytic unit often has to be overdesigned by using a larger reactor and more catalyst to such a degree that it can have some flexibility to handle a wide range of CH₃Br slippage from the synthesis reactor.

Thus, although progress has been made in the conversion of lower molecular weight alkanes to higher molecular weight hydrocarbons, there remains a need for processes that are more efficient, economic, and safe to operate.

### SUMMARY

To achieve the foregoing and other objects, and in accordance with the purposes of the present invention, as embodied and broadly described herein, one embodiment of the present invention is a process that comprises reacting at least gaseous alkanes and a halogen to produce at least a halogenation product stream, wherein the halogenation product stream comprises alkyl halides, hydrogen halide, and unreacted alkanes. The process may further comprise reacting at least a portion of the alkyl halides from the halogenation product stream in the presence of a catalyst to produce at least a synthesis product stream, wherein the synthesis product stream comprises unreacted methyl halide, higher molecular weight hydrocarbons, and hydrogen halide. The process may further comprise separating the synthesis product stream into at least a first stream comprising hydrocarbons having five or more carbons, a second stream comprising unreacted methyl halide, and a third stream comprising hydrogen halide and hydrocarbons having one to four carbons.

Another embodiment of the present invention is a process that comprises reacting at least gaseous alkanes and bromine in a bromination reactor to produce at least a bromination product stream, wherein the bromination product stream comprises alkyl bromides, hydrogen bromide, and unreacted alkanes. The process may further comprise separating the bromination product stream into at least a gaseous stream and a liquid alkyl bromides stream, wherein the gaseous stream comprises hydrogen bromide and unreacted alkanes, and wherein the liquid alkyl bromides stream comprises alkyl bromides. The process may further comprise separating the liquid alkyl bromides stream into at least a monobromides stream and a polybromides stream, wherein the monobromides stream comprises monobrominated alkanes, and wherein the polybromides stream comprises polybrominated alkanes. The process may further comprise reacting at least a portion of the monobrominated alkanes from the monobromides stream in a synthesis reactor in the presence of a catalyst to produce at least a synthesis product stream, wherein the synthesis product stream comprises unreacted methyl bromide, higher molecular weight hydrocarbons, and hydrogen bromide. The process may further comprise separating the synthesis product stream into at least a first stream comprising hydrocarbons having five or more carbons, a second stream comprising unreacted methyl bromide, and a third stream comprising hydrogen bromide and hydrocarbons having one to four carbons.

Yet another embodiment of the present invention is a system that comprises a halogenation reactor configured for reaction of at least gaseous alkanes and a halogen to produce at least a halogenation product stream, wherein the halogenation product stream comprises alkyl halides, hydrogen halide, and unreacted alkanes. The system further may comprise a synthesis reactor in fluid communication with the halogenation reactor configured for reaction of at least a portion of the alkyl halides from the halogenation product stream in the presence of a catalyst to produce a synthesis product stream, wherein the synthesis product stream comprises methyl halide, higher molecular weight hydrocarbons, and hydrogen halide. The system further may comprise a dehalogenation system in fluid communication with the synthesis reactor configured for separation of for separating the synthesis product stream into at least a first stream comprising hydrocarbons having five or more carbons, a second stream comprising methyl halide, and a third stream comprising hydrogen halide and hydrocarbons having one to four carbons.

### BRIEF DESCRIPTION OF THE DRAWINGS

These drawings illustrate certain aspects of some of the embodiments of the present invention and should not be used to limit or define the invention.

FIG. 1 is a schematic view of a process for the conversion of lower molecular weight alkanes to higher molecular weight hydrocarbons that includes a debromination system for the removal of residual CH₃Br from the synthesis product stream in accordance with embodiments of the present invention.

FIG. 2 is a schematic view of another embodiment of a process for the conversion of lower molecular weight alkanes to higher molecular weight hydrocarbons that includes a debromination system for the removal of residual CH₃Br from the synthesis product stream with fractionation of brominated hydrocarbons upstream of the synthesis reactor.

FIG. 3 is a schematic view of another embodiment of a process for the conversion of lower molecular weight alkanes to higher molecular weight hydrocarbons that includes a debromination system for the removal of residual CH₃Br from the synthesis product stream configured to incorporate a shift reactor for reducing the content of polybrominated alkanes fed to the synthesis reactor.

FIG. 4 is a schematic view of another embodiment of a process for the conversion of lower molecular weight alkanes to higher molecular weight hydrocarbons that includes a debromination system for the removal of residual CH₃Br from the synthesis product stream with recycle of light end hydrocarbons to produce light end bromides for an additional feed to the synthesis reactor.

FIG. 5 is a schematic view of a debromination system in accordance with embodiments of the present invention.

### DETAILED DESCRIPTION OF SPECIFIC EMBODIMENTS

Embodiments of the present invention are directed to processes and systems for converting lower molecular weight alkanes to higher molecular weight hydrocarbons that include recovery of halogenated hydrocarbons (e.g., CH₃Br) from higher molecular weight hydrocarbon products.

There may be many potential advantages to the methods and systems of the present invention, only some of which are alluded to herein. One of the many potential advantages of the embodiments of the systems and methods of the present invention is that unconverted CH₃Br can be removed and recovered from the higher molecular weight hydrocarbon products downstream of the synthesis reactor, thus allowing for recycling of CH₃Br for upstream use while minimizing bromine loss from CH₃Br. In addition, by recovery and recycle of CH₃Br, the carbon and hydrogen constituents of CH₃Br may not be lost to carbon monoxide, carbon dioxide, and water, which would represent yield loss and also possibly generate highly corrosive aqueous/alcoholic hydrobromic acid stream, complicating the process metallurgy. Furthermore, because halogenated hydrocarbons are typically toxic compounds presenting a hazard risk, the removal and recovery of CH₃Br after the synthesis reactor should confine the presence of these toxic compounds to a relatively small area of the process. Yet another potential advantage of the embodiments and systems of the present invention is that embodiments of the methods for removing and recovering CH₃Br do not involve a catalytic reaction. Yet another potential advantage is that embodiments of the methods for removing and recovering CH₃Br fractionate the synthesis product stream into three streams: a first stream comprising HBr, a second stream comprising CH₃Br, and a third stream comprising C5+ hydrocarbons.

The term “higher molecular weight hydrocarbons,” as used herein, refers to hydrocarbons comprising a greater number of carbon atoms than one or more components of the feedstock. For example, natural gas is typically a mixture of light hydrocarbons, predominately methane, with lesser amounts of ethane, propane, and butane, and even smaller amounts of longer chain hydrocarbons such as pentane, hexane, etc. When natural gas is used as a feedstock, higher molecular weight hydrocarbons produced in accordance with embodiments of the present invention may include a hydrocarbon comprising C2 and longer hydrocarbon chains, such as propane, butane, C5+ hydrocarbons, aromatic hydrocarbons, and mixtures thereof. In some embodiments, part or all of the higher molecular weight hydrocarbons may be used directly as a product (e.g., LPG, motor fuel, etc.). In other instances, part or all of the higher molecular weight hydrocarbons may be used as an intermediate product or as a feedstock for further processing. In yet other instances, part or all of the higher molecular weight hydrocarbons may be further processed, for example, to produce gasoline grade fuels, diesel grade fuels, and fuel additives. In some embodiments, part or all of the higher molecular weight hydrocarbons obtained by the processes of the present invention can be used directly as a motor gasoline fuel having a substantial aromatic content, as a fuel blending stock, or as feedstock for further processing such as an aromatic feed to a process producing aromatic polymers, such as polystyrene or related polymers.

The end use of the higher molecular weight hydrocarbons may depend on the particular catalyst employed for the coupling reaction carried out in the synthesis reactor discussed below, as well as the operating parameters employed in the process. Other uses will be evident to those skilled in the art with the benefit of this disclosure.

The term “alkyl bromides,” as used herein, refers to mono-, di-, and tri-brominated alkanes, and combinations of these. Polybrominated alkanes include di-brominated alkanes, tri-brominated alkanes and mixtures thereof. These alkyl bromides may then be reacted over suitable catalysts so as to form higher molecular weight hydrocarbons.

The term “lower molecular weight alkanes,” as used herein, refers to methane, ethane, propane, butane, pentane or mixtures of two or more of these individual alkanes. Lower molecular weight alkanes may be used as a feedstock for the methods described herein. The lower molecular weight alkanes may be from any suitable source, for example, any source of gas that provides lower molecular weight alkanes, whether naturally occurring or synthetically produced. Examples of sources of lower molecular weight alkanes for use in the processes of the present invention include, but are not limited to, natural gas, coal-bed methane, regasified liquefied natural gas, gas derived from gas hydrates and/or clathrates, gas derived from anaerobic decomposition of organic matter or biomass, gas derived in the processing of tar sands, and synthetically produced natural gas or alkanes. Combinations of these may be suitable as well in some embodiments. In some embodiments, it may be desirable to treat the feed gas to remove undesirable compounds, such as sulfur compounds and carbon dioxide.

Suitable sources of bromine that may be used in various embodiments of the present invention include, but are not limited to, elemental bromine, bromine salts, aqueous hydrobromic acid, metal bromide salts, and the like. Combinations may be suitable, but as recognized by those skilled in the art, using multiple sources may present additional complications.

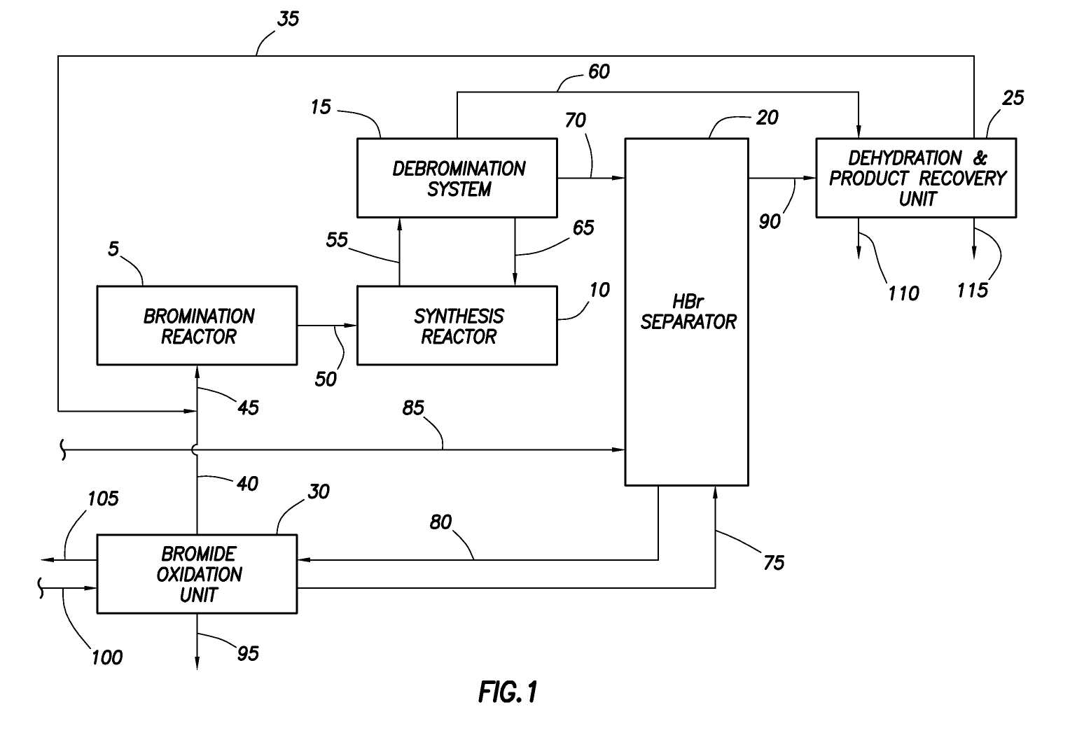

FIG. 1 is a schematic diagram illustrating a bromine-based process for the conversion of lower molecular weight alkanes to higher molecular weight hydrocarbons that includes recovery of residual CH₃Br from higher molecular weight hydrocarbon products in accordance with embodiments of the present invention. As illustrated, embodiments of the process may include a bromination reactor 5 for brominating lower molecular alkanes to form alkyl bromides, a synthesis reactor 10 for production of higher molecular weight hydrocarbons from the alkyl bromides, a debromination system 15 for recovery of residual CH₃Br from the higher molecular weight hydrocarbons, an HBr separator 20 for recovery of HBr generated in the process, a dehydration and product recovery unit 25 for product purification and feedstock recycle, and a bromide oxidation unit 30 for recovery of elemental bromine.

In the illustrated embodiment, a gas stream 35 comprising lower molecular weight alkanes (which, in some embodiments, may include a mixture of feed gas plus recycle gas) and a bromine stream 40 may be combined and introduced into the bromination reactor 5. In the illustrated embodiment, the gas stream 35 and the bromine stream 40 are premixed to form a bromination feed gas stream 45, which is fed to the bromination reactor 5. In an alternative embodiment (not illustrated), the gas stream 35 and the bromine stream 40 may be combined in the bromination reactor 5. The gas stream 35 and the bromine stream 40 may be allowed to react in the bromination reactor 5 to form a bromination product stream 50 that comprises alkyl bromides, HBr vapor, and unreacted alkanes. The bromination product stream 50 may be withdrawn from the bromination reactor 5.

In the bromination reactor 5, the lower molecular weight alkanes in the gas stream 35 may be reacted exothermically with the bromine in the bromine stream 40, for example, at a temperature in the range of about 250° C. to about 600° C., and at a pressure in the range of about 1 bar gauge (“barg”) to about 50 barg to produce gaseous alkyl bromides and HBr. In an embodiment, the operating pressure of the bromination reactor 5 may range from about 20 barg to about 40 barg. In some embodiments, the feeds to the bromination reactor 5 may be pre-heated to a temperature of about 250° C. to about 400° C., for example, in an inlet pre-heater zone. It should be understood that the upper limit of the operating temperature range can be greater than the upper limit of the reaction initiation temperature range to which the bromination feed gas stream 45 may be heated due to the exothermic nature of the bromination reaction. The bromination reaction may be a non-catalytic (thermal) or a catalytic reaction as will be appreciated by those of ordinary skill in the art. Bromination of alkanes is described in more detail in U.S. Pat. No. 7,674,941, the disclosure of which is incorporated herein by reference. In the case of methane, it is believed that the formation of multiple brominated compounds occurs in accordance with the following general overall reaction:

aCH₄(g)+bBr₂(g)→cCH₃Br(g)+dCH₂Br₂(g)+eCHBr₃(g)+fCBr₄(g)+xHBr(g)

The methane/bromine molar ratio of the feed introduced to the bromination reactor 5 may be at least about 2.5:1, in some embodiments. In alternative embodiments, a larger excess of methane (e.g., about 3:1 to about 10:1) may be used in order to achieve desirable selectivity of CH₃Br and reduce the formation of soot, as CH₃Br is more rapidly brominated than methane under free radical conditions. The C2+ alkanes entering the bromination reactor 5 are known to more rapidly form polybrominated alkanes and coke/soot, as they are much more easily brominated than methane. Accordingly, in some embodiments, the C2+ alkane content entering the bromination reactor 5 can be controlled by treating the natural gas feed stream 85 or its mixture with the hydrocarbon products formed in the synthesis reactor 10 using any suitable means, such as cryogenic separation. In some embodiment, the C2+ alkane concentration in the total alkanes fed to the bromination reactor 5 is less than about 10 mole % in one embodiment, less than about 1 mole % in another embodiment, less than about 0.2 mole % in another embodiment, and less than about 0.1 mole % in yet another embodiment.

In some embodiments, the bromination product stream 50 may be fed to the synthesis reactor 10. In the synthesis reactor 10, the alkyl bromides may be reacted over a suitable catalyst under sufficient conditions via a catalytic coupling reaction to produce higher molecular weight hydrocarbons and additional HBr vapor. In some embodiments, a fixed-bed reactor may be used. In alternative embodiments, a fluidized-bed reactor may be used. Those of ordinary skill in the art will appreciate, with the benefit of this disclosure, that the particular higher molecular weight hydrocarbons produced will be dependent, for example, upon the catalyst employed, the composition of the alkyl bromides introduced, and the exact operating parameters employed. Catalysts that may be employed in the synthesis reactor 10 include synthetic crystalline alumino-silicate catalysts as will be recognized by those of ordinary skill in the art. Formation of higher molecular weight hydrocarbons from reaction of brominated hydrocarbons is described in more detail in U.S. Pat. No. 7,674,941.

As illustrated, a synthesis product stream 55 comprising higher molecular weight hydrocarbons may be withdrawn from the synthesis reactor 10 and fed to the debromination system 15. The higher molecular weight hydrocarbons in the synthesis product stream 55 may comprise C2-C4 hydrocarbons and C5+ heavy-end hydrocarbons. Because complete conversion of the alkyl bromides will likely not occur in the synthesis reactor 10, the synthesis product stream 55 will likely also comprise residual alkyl bromides. The synthesis product stream 55 further may comprise methane (e.g., residual methane from gas stream 35 and an unintended amount of methane produced in the synthesis reactor 10) and HBr vapor (e.g., produced in the bromination reactor 5 and the synthesis reactor 10).

In the debromination system 15, the synthesis product stream 55 may be separated into a C5+ stream 60 comprising pentane and heavier hydrocarbons, a CH₃Br stream 65 comprising the residual CH₃Br, and an HBr/hydrocarbon stream 70 comprising methane, C2-C4 hydrocarbons, and HBr. The CH₃Br stream 65 may also comprise a quantity of hydrocarbons, e.g., C3-C4 hydrocarbons. In some embodiments, the C5+ stream 60 may be essentially free of CH₃Br, for example, containing CH₃Br in an amount of less than about 10 molar parts per million (“mppm”) of the C5+ stream 60 and, alternatively, less than about 1 mppm. As illustrated, the C5+ stream 60 may bypass the HBr separator 20 and be routed to the dehydration and product recovery unit 25. It should be understood that residual alkyl bromides heavier than CH₃Br may be separated from the C5+ stream 60 in the dehydration and product recovery unit 25, in accordance with certain embodiments. In some embodiments, the CH₃Br stream 65 may be essentially free of C5+ hydrocarbons, for example, containing C5+ hydrocarbons in an amount of less than about 100 mppm of the CH₃Br stream 65 and, alternatively, less than about 10 mppm. As illustrated, the CH₃Br stream 65 may be recycled back to the synthesis reactor 10 for reaction over the catalyst to produce higher molecular weight hydrocarbons and HBr vapor. The illustrated embodiment shows the HBr/hydrocarbon stream 70 being fed to the HBr separator 20 for separation of the HBr from the methane and C2-C4 hydrocarbons.

In the HBr separator 20, any of a variety of different suitable techniques may be used for separation of HBr, including, but not limited to, the techniques disclosed in U.S. Pat. No. 7,674,941. Non-limiting examples of techniques for HBr separation include absorption of HBr into an aqueous solution or adsorption of HBr on a metal oxide. In the illustrated embodiment, the HBr/hydrocarbon stream 70 may be contacted with recirculating aqueous solution 75 in the HBr separator 20 to recover HBr from the hydrocarbons by absorbing it into the aqueous solution. The resultant aqueous solution comprising HBr dissolved therein may be removed from the HBr separator 20 via aqueous HBr stream 80.

As illustrated, natural gas feed stream 85 may enter the HBr separator 20 for recovery of hydrocarbons or other purposes. For example, the natural feed gas stream 85 may strip out any residual hydrocarbons in the resultant aqueous solution comprising HBr dissolved therein, depending on the solubility of the hydrocarbons in the aqueous solution at the operating conditions. While not illustrated by FIG. 1, the natural gas feed stream 85 may alternatively be fed directly to the dehydration and product recovery unit 25 for removal of C2+ hydrocarbons. While the present embodiment describes the use of natural gas feed stream 85, as discussed above, embodiments of the present invention encompass the use of other feedstocks of lower molecular weight alkanes.

The aqueous HBr stream 80 may withdrawn from the HBr separator 20 and routed to the bromide oxidation unit 30, in some embodiments, to convert the dissolved HBr to elemental bromine using, for example, air or oxygen and to regenerate the aqueous solution for reuse in the HBr separator 20. The regenerated aqueous solution may then be recirculated to the HBr separator 20 via recirculating aqueous solution 75. The bromine may then be treated sufficiently and sent to the bromination reactor 5 via bromine stream 40. In some embodiments, the bromine that is feed into the bromination reactor 5 may be dry bromine in that the bromine is substantially water-free. Effluent water 95 may also be removed from the bromide oxidation unit 30. Line 100 may be used to supply the oxygen or air fed to the bromide oxidation unit 30. Residual oxygen or spent air may be removed from the oxidation unit via line 105.

In some embodiments, hydrocarbon stream 90 comprising an unintended amount of methane produced in the synthesis reactor 10, higher molecular weight hydrocarbons, and the feed gas may be withdrawn from the HBr separator 20. The hydrocarbon stream 90 may be substantially HBr free, in accordance with embodiments of the present invention, for example, containing less than about 1 mppm HBr and alternatively less than 0.1 mppm HBr.

As illustrated, the C5+ stream 60 from the debromination system 15 and the hydrocarbon stream 90 from the HBr separator 20 may be routed to dehydration and product recovery unit 25 wherein water may be removed from the remaining constituents, higher molecular weight hydrocarbons may be recovered as liquid hydrocarbon products, and lower molecular weight hydrocarbons (e.g., methane, ethane, etc.) may be recycled to the bromination reactor 5. Any suitable method of dehydration and product recovery may be used, including, but not limited to, solid-bed desiccant adsorption followed by refrigerated condensation, cryogenic separation, or circulating absorption oil or some other suitable solvent. As illustrated, water may be removed via water stream 110. A liquid hydrocarbon product stream 115 comprising higher molecular weight hydrocarbons may be withdrawn for use as a fuel, a fuel blend, or for further petrochemical or fuel processing, for example.

In the illustrated embodiment, the gas stream 35 comprising methane from the dehydration and product recovery unit 25 (which may be a mixture of feed gas plus recycled gas) may be fed to the bromination reactor 5. It should be understood that the gas stream 35 may also comprise some C2+ alkanes so long as the C2+ content fed to the bromination reactor 5 is less than a predetermined value.

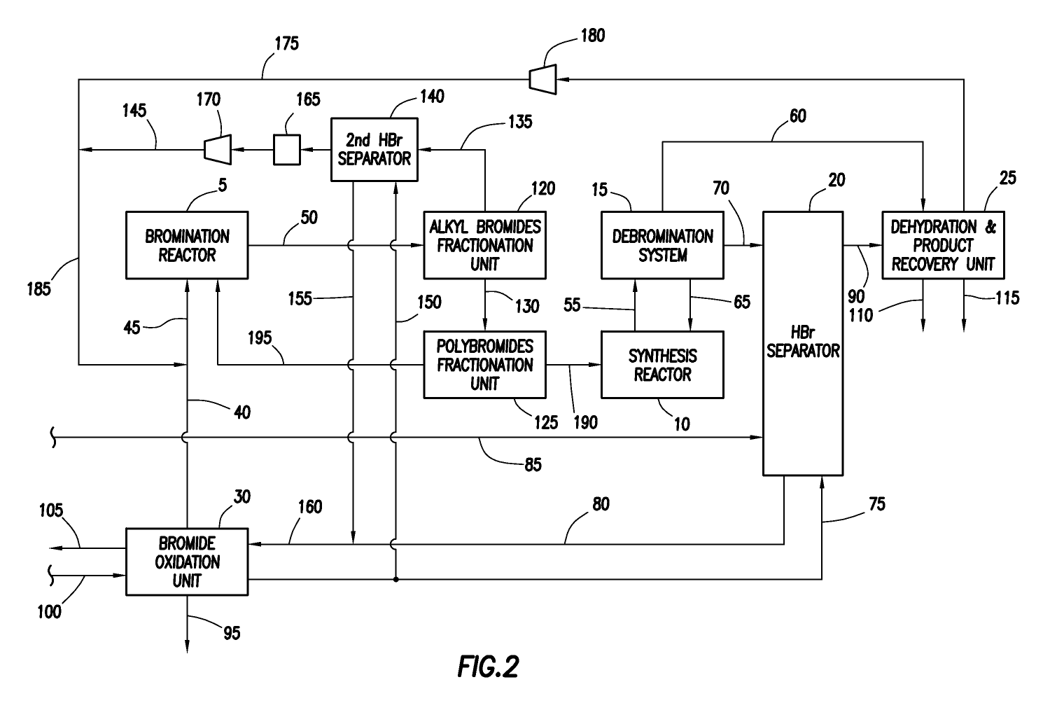

Referring now to FIG. 2, a bromine-based process is illustrated for the conversion of lower molecular weight alkanes to higher molecular weight hydrocarbons that includes a debromination system for the removal of residual CH₃Br from the synthesis product stream with fractionation of brominated hydrocarbons upstream of the synthesis reactor in accordance with embodiments of the present invention. The illustrated embodiment is similar to that illustrated by FIG. 1 except that there are additional units between the bromination reactor 5 and the synthesis reactor 10. As illustrated, the bromine-based system further includes an alkyl bromides fractionation unit 120 for separation of unreacted alkanes and HBr from the brominated alkanes and a polybromides fractionation unit 125 for separation of polybrominated alkanes from monobrominated alkanes.

As illustrated, the bromination product stream 50 comprising alkyl bromides, HBr vapor, and unreacted alkanes can be withdrawn from the bromination reactor 5 and fed to an alkyl bromides fractionation unit 120. In the alkyl bromides fractionation unit 120, the bromination product stream 50 may be separated into a liquid alkyl bromides stream 130 comprising CH₃Br and other heavier alkyl bromides and a gaseous alkane/HBr stream 135 comprising unreacted alkanes (e.g., methane) and HBr. The liquid alkyl bromides stream 130 may comprise monobrominated alkanes (e.g., CH₃Br and other heavier monobrominated alkanes) and polybrominated alkanes (e.g., CH₂Br₂ and other heavier polybrominated alkanes), and the gaseous alkane/HBr stream 135 may comprise unreacted alkanes and HBr.

In some embodiments, the gaseous alkane/HBr stream 135 comprising the unreacted alkanes and HBr may be withdrawn from the alkyl bromides fractionation unit 120 and fed to a second HBr separator 140. In the second HBr separator 140, any of a variety of different suitable techniques may be used to produce a recycle gas stream 145 by separation of HBr, including, but not limited to, the techniques disclosed in U.S. Pat. No. 7,674,941. Non-limiting examples of techniques for HBr separation include absorption HBr into an aqueous solution or adsorption of HBr on a metal oxide. In some embodiments, the HBr can be recovered from the unreacted alkanes by adsorbing the HBr into an aqueous solution using, for example, a packed column or other suitable containing device. The aqueous solution may be fed to the second HBr separator via second recirculating aqueous stream 150.

The second HBr separator 140 can operate at a different, and preferably, higher pressure than the HBr separator 20 which recovers HBr from the HBr/hydrocarbon stream 70 from the debromination system 15. For example, the second HBr separator 140 can operate at a pressure that is at least about 3 bars higher than the HBr separator 20. In some embodiments, the second HBr separator 140 may operate at a pressure of about 5 barg to about 50 barg while the HBr separator 20 operates at a pressure a pressure of about 2 barg to about 47 barg.

The resultant aqueous solution comprising HBr dissolved therein may be removed from the second HBr separator 140 via second aqueous HBr stream 155, in accordance with embodiments of the present invention. The second aqueous HBr stream 155 may be combined with aqueous HBr stream 80 from the HBr separator 20 and fed to the bromide oxidation unit 30 via line 160 to produce elemental bromine and regenerate the aqueous solutions for reuse in the HBr separator 20 and the second HBr separator 140. While FIG. 2 illustrates combination of the aqueous HBr stream 80 and the second aqueous HBr stream 155 prior to entering the bromide oxidation unit 30, embodiments (not illustrated) may include separately feeding the aqueous HBr streams 80, 155 to the bromide oxidation unit 30.

The recycle gas stream 145 containing the alkanes (e.g., methane) separated from HBr in the second HBr separator 140 may be fed to a second dehydrator 165 for the removal of water and then recycle compressor 170 before being combined with feed gas stream 175 from the dehydration and product recovery unit 25. As illustrated, the feed gas stream 175 may be routed to feed compressor 180 before combination with the recycle gas stream 145. The feed/recycle gas stream 185 comprising a mixture of the recycle gas stream 145 and the feed gas stream 175 may be combined with bromine stream 40 and fed to the bromination reactor 5 via bromination feed gas stream 45. While FIG. 2 illustrates combination of the recycle gas stream 145 and the feed gas stream 175 prior to entering the bromination reactor 5, embodiments (not illustrated) may include separately feeding the recycle gas stream 145 and the feed gas stream 175 to the bromination reactor 5.

In the illustrated embodiment, the unreacted alkanes separated from the alkyl bromides in the alkyl bromides fractionation unit 120 are only circulating through the bromination reactor 5, the alkyl bromides fractionation unit 120, the second HBr separator 140, and the second dehydrator 160, enduring much less pressure drop by avoiding circulation through the entire system as disclosed in the process schemes used heretofore. As a result, the increase in compression cost for using a large excess of methane or high methane-to-bromine ratio in the bromination reactor 5 can be minimized by incorporation of embodiments of the present invention.

In some embodiments, the liquid alkyl bromides stream 130 may be withdrawn from the alkyl bromides fractionation unit 120 and fed to the polybromides fractionation unit 125. Prior to entering the polybromides fractionation unit 125, the liquid alkyl bromides stream 130 may be pumped to a higher pressure or let down to a lower pressure, as desired for a particular application. In some embodiments, the polybromides bromides fractionation unit 125 may have an operating pressure from about 1 barg to about 20 barg, for example, to minimize reboiler temperature (e.g., <250° C., alternatively, <200° C.) required for the polybromides fractionation while allowing the use of an inexpensive cooling medium (e.g., cooling water or air cooler) for the overhead condenser. In the polybromides fractionation unit 125, the liquid alkyl bromides stream 130 may be separated into a monobromides stream 190 comprising CH₃Br and other heavier monobrominated alkanes and a polybromides stream 195 comprising CH₂Br₂ and other heavier polybrominated alkanes. In the illustrated embodiment, the polybromides stream 195 is returned to the bromination reactor 5 for reproportionating with lower molecular weight alkanes to produce a quantity of monobrominated alkanes in addition to those produced from reaction of the bromine and lower molecular alkanes. While not illustrated by FIG. 2, reproportionation of the polybrominated alkanes in the polybromides stream 195 may occur in a separate reactor from the bromination reactor 5 in accordance with alternative embodiments.

In some embodiments, the monobromides stream 190 comprising CH₃Br and other heavier monobrominated alkanes may be vaporized and fed to the synthesis reactor 10. In the synthesis reactor 10, the monobrominated alkanes may be reacted over a suitable catalyst under sufficient conditions via a catalytic coupling reaction to produce higher molecular weight hydrocarbons and additional HBr vapor. By separating some or all of the polybrominated alkanes from the feed to the synthesis reactor 10, coke formation in the synthesis reactor 10 may be reduced. By reducing coke formation in the synthesis reactor 10, the deactivation rate of the catalyst may be reduced. Due to this reduction in the deactivation rate, a fixed-bed reactor may be suitable, in some embodiments, even for commercial-scale production. In alternative embodiments, a fluidized-bed reactor may be used.

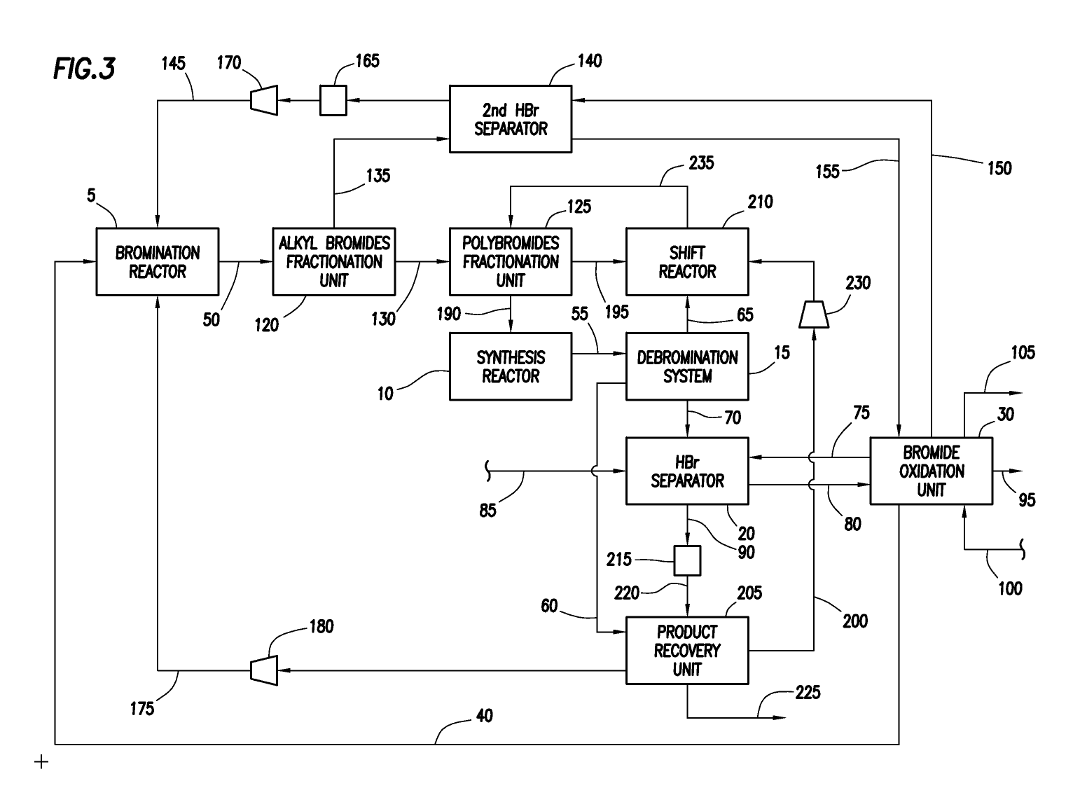

Referring now to FIG. 3, a bromine-based process is illustrated for the conversion of lower molecular weight alkanes to higher molecular weight hydrocarbons that includes a debromination system for the removal of residual CH₃Br from the synthesis product stream configured to incorporate a shift reactor for reducing the content of polybrominated alkanes fed to the synthesis reactor in accordance with embodiments of the present invention. The illustrated embodiment is similar to that illustrated by FIG. 2 except that a light ends product stream 200 comprising C2-C4 hydrocarbons is specified as an additional product from product recovery unit 205. As illustrated, the light ends product stream 200 may be recycled to reproportionate polybrominated alkanes in a shift reactor 210, producing a quantity of monobrominated alkanes in addition to those produced in the bromination reactor 5. It should be understood that when the light ends product stream 205 is specified, the feed gas stream 175 routed to the bromination reactor 5 can contain substantially pure methane, in some embodiments, in that the C2+ alkane concentration in the feed gas stream 175 may be less than about 1 mole %, in one embodiment, and less than about 0.1 mole %, in another embodiment.

In the illustrated embodiment, hydrocarbon stream 90 comprising an unintended amount of methane produced in the synthesis reactor 10, C2-C4 hydrocarbons from the debromination system 15, and the feed gas may be withdrawn from the HBr separator 20 and routed to a first dehydrator 215 for removal of water. The dehydrated hydrocarbon stream 220 may be withdrawn from the first dehydrator 215 and routed to the product recovery unit 205 for recovery of a heavy ends product stream 225 comprising C5+ hydrocarbons, a light ends product stream 200 comprising C2-C4 hydrocarbons, and a feed gas stream 175 comprising methane. Any suitable method of dehydration and product recovery may be used, including, but not limited to, solid-bed desiccant adsorption followed by refrigerated condensation, cryogenic separation, or circulating absorption oil or some other solvent.

The feed gas stream 175 comprising methane from the product recovery unit 205 may be fed to the bromination reactor 5 via the feed compressor 180. It should be understood that the feed gas stream 175 may also comprise some C2+ alkanes so long as the C2+ content of the alkanes (e.g., feed gas stream 175+ recycle gas stream 145) fed to the bromination reactor 5 is less than a predetermined value. While FIG. 3 illustrates the feed gas stream 175 and the recycle gas stream 145 as separate feeds to the bromination reactor 5, it should be understood that embodiments include premixing the feed gas stream 175 and the recycle gas stream 145 prior to feeding the bromination reactor 5.

As illustrated, the light ends product stream 200 comprising C2-C4 hydrocarbons may be fed to the shift reactor 210 via a light ends recycle compressor 230. The polybromides stream 195 from the polybromides fractionation unit 125 comprising CH₂Br₂ and other heavier polybrominated alkanes may also be fed to the shift reactor 210. The CH₃Br stream 65 comprising residual CH₃Br and hydrocarbons (e.g., C3-C4 hydrocarbons) from the debromination system 15 may also be fed to the shift reactor 20. In some embodiments, the feeds may be vaporized prior to their introduction into the shift reactor 210. In the shift reactor 210, at least a portion of the polybrominated alkanes in the polybromides stream 195 can be reproportionated into monobrominated alkanes, thus increasing the content of monobrominated alkanes in the feed to the synthesis reactor 10. This shift reaction occurs, for example, by reaction of the C2-C4 hydrocarbons in the light ends product stream 200 and the C3-C4 hydrocarbons in the CH₃Br stream 65 with the polybrominated alkanes to form monobrominated alkanes, such as CH₃Br, ethyl bromide (C₂H₅Br), propyl bromide (C₃H₇Br), and the like. In some embodiments, the shift reaction may proceed thermally without a catalyst. In another embodiment, the shift reaction may be a catalytic reaction. Example techniques for reproportionation of polybrominated alkanes via a shift reaction are described in more detail in U.S. Pat. No. 7,674,941.

In the illustrated embodiment, a reproportionated alkyl bromides stream 235 comprising monobrominated alkanes, unreacted C2-C4 hydrocarbons, and unconverted polybromides may be withdrawn from the shift reactor 210 and routed back to the polybromides fractionation unit 125. As previously discussed, the polybromides fractionation unit 125 also receives a liquid alkyl bromides stream 130 as a feed from the alkyl bromides fractionation unit 120. In the illustrated embodiment, the polybromides fractionation unit 125 separates the reproportionated alkyl bromides stream 235 and liquid alkyl bromides stream 130 into a monobromides stream 190 and a polybromides stream 195. In one embodiment, the monobromides stream 190 may be fed to the synthesis reactor 10 for reaction over a suitable catalyst to produce higher molecular weight hydrocarbons. As illustrated, the polybromides stream 195 may be fed to the shift reactor 210 for another round of reproportionation.

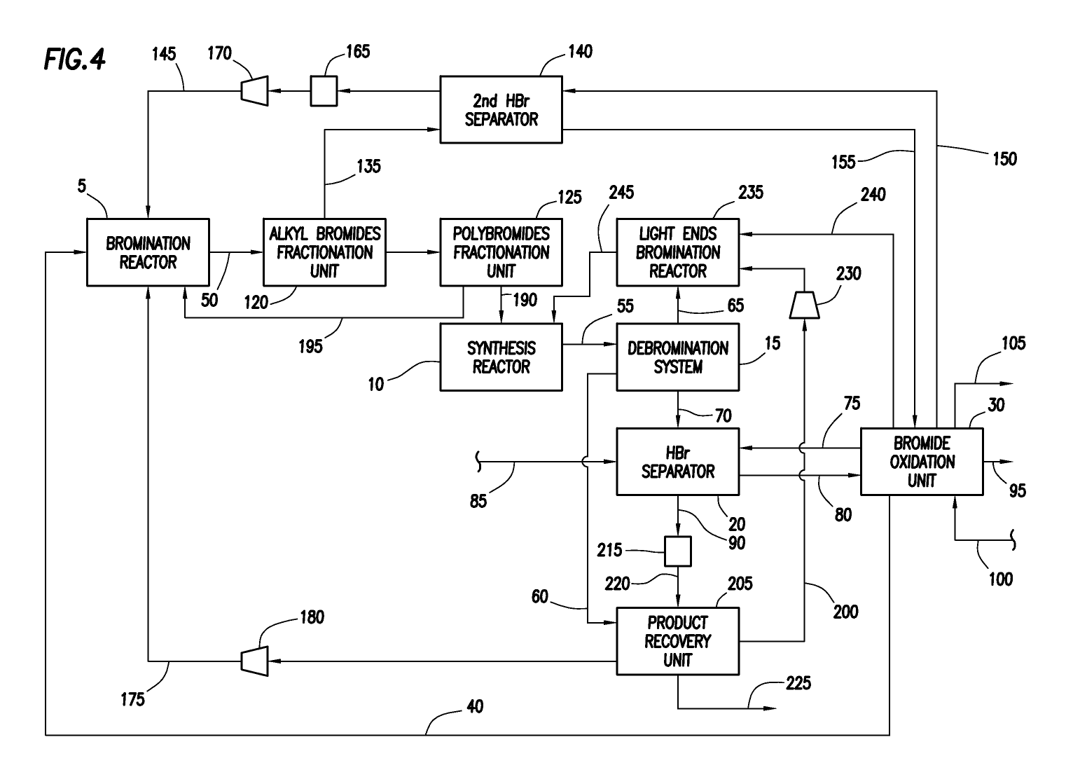

Referring now to FIG. 4, a bromine-based process is illustrated or the conversion of lower molecular weight alkanes to higher molecular weight hydrocarbons that includes a debromination system for the removal of residual CH₃Br from the synthesis product stream with recycle of C2-C4 hydrocarbons to produce light end bromides for an additional feed to the synthesis reactor in accordance with embodiments of the present invention. The illustrated embodiment is similar to that illustrated by FIG. 3 expect that the light ends product stream 200 comprising C2-C4 hydrocarbons is recycled to a light ends bromination reactor 235 to produce C2+ bromides, preferably C2+ monobromides, for additional feed to the synthesis reactor 10.

As illustrated, the light ends product stream 200 may be fed to the light ends bromination reactor 235 via light ends recycle compressor 230. In the light ends bromination reactor 235, the light end hydrocarbons may be allowed to react with bromine fed to the reactor 235 via line 240 to form products that comprise C2+ alkyl bromides, HBr vapor, and unreacted light end hydrocarbons. The CH₃Br stream 65 comprising residual CH₃Br and hydrocarbons (e.g., C3-C4 hydrocarbons) from the debromination system 15 may also be fed to the shift reactor 20 for reaction with the bromine in line 240 to form C2+ alkyl bromides (e.g., C2+ monobromides).

In some embodiments, the light ends bromination reactor 235 may operate at milder conditions than the bromination reactor 5. For example, the light ends bromination reactor 235 may operate at a temperature in the range of about 200° C. to about 500° C., alternatively about 235° C. to about 450° C., and alternatively about 250° C. to about 425° C. By way of further example, the light ends bromination reactor 235 may operate at a pressure in the range of about 1 barg to about 80 barg, alternatively about 10 barg to about 50 barg, and alternatively about 20 barg to about 40 barg. In one embodiment, the light ends bromination reactor 235 may operate at a temperature in the range of about 250° C. to about 425° C., and at a pressure in the range of about 15 barg to about 35 barg while the bromination reactor 5 may operate at a temperature in the range of about 350° C. to about 500° C. and a pressure of about 25 barg to about 40 barg.

The light ends bromination reactor effluent 245 that contains the C2+ alkyl bromides, HBr vapor, and unreacted light end hydrocarbons may be withdrawn from the light ends bromination reactor 235 and fed to the synthesis reactor 10. In the synthesis reactor 10, the C2+ alkyl bromides may react over a suitable catalyst to produce higher molecular weight hydrocarbons. While light ends bromination reactor effluent 245 and the monobromides stream 190 from the polybromides fractionation unit 120 comprising CH₃Br and other heavier monobrominated alkanes are illustrated as separate feeds to the synthesis reactor 10, it should be understood that present embodiments encompass processes in which these streams are combined prior to feeding the synthesis reactor 10. The C2+ alkyl bromides in the light ends bromination reactor effluent 245 may be less contributive to formation of coke in the synthesis reactor 10 than C1 polybromides; therefore, the light ends bromination reactor effluent 245 may not require further treatment prior to entering the synthesis reactor 10 in some embodiments. In alternative embodiments (not illustrated), the light ends bromination reactor effluent 245 may be fractionated, for example, to separate polybromides from the feed to the synthesis reactor 10. Fractionation of the light ends bromination reactor effluent 245 may be desirable, for example, where strict control of alkyl bromides (including C2+ polybromides) is necessary to achieve a minimum and steady coke formation rate and/or a desirable and steady product selectivity profile.

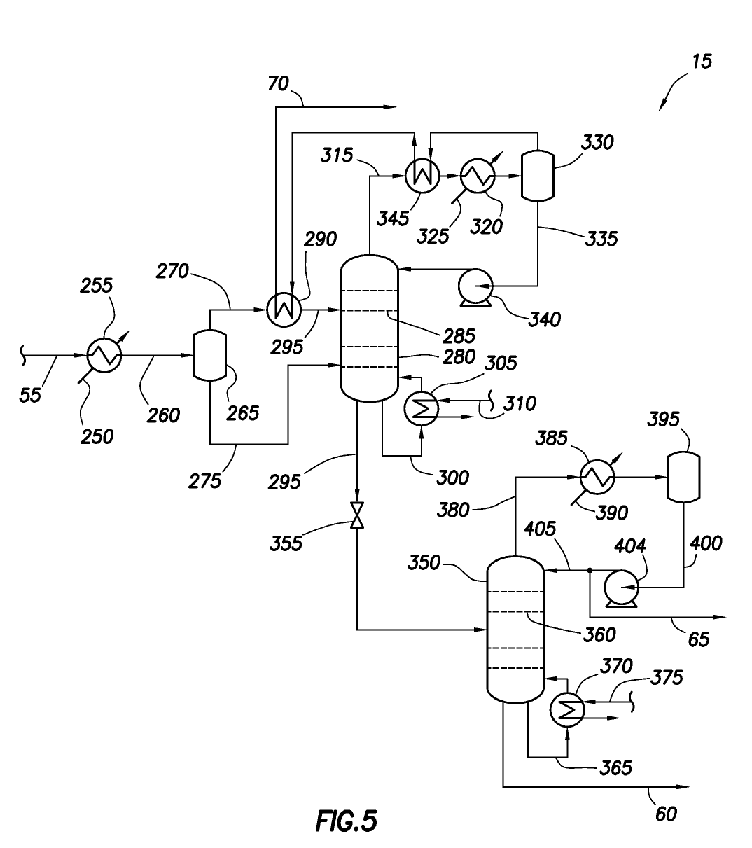

Referring now to FIG. 5, a debromination system 15 is illustrated in accordance with embodiments of the present invention. In the illustrated embodiment, the debromination system 15 separates the synthesis product stream 55 into a C5+ stream 60, a CH₃Br stream 65, and an HBr/hydrocarbon stream 70.

In the illustrated embodiment, the synthesis product stream 55 comprising higher molecular weight hydrocarbons (e.g., C2+ hydrocarbons), methane (e.g., residual methane and/or methane produced in the synthesis reactor 10), and residual CH₃Br can first be cooled. As illustrated, the synthesis product stream 55 may be cooled, for example, to a temperature of about 33° C. to about 43° C., by exchanging heat with water stream 250 in water-cooled heat exchanger 255. It should be understood that a cooling medium other than water stream 250 may be used in some embodiments, for example, to obtain a lower temperature (e.g., about −10° C. to about 33° C.) for the cooled synthesis product stream 260 exiting the heat exchanger 255. While not illustrated, the synthesis product stream 55 may be cooled, in some embodiments, by exchanging heat with one or more other process streams in one or more cross heat exchangers, prior to water cooling. The cooled synthesis product stream 260, which partially condenses in the water-cooled heat exchanger 255, may then be sent, in one embodiment, to a feed separator 265 (e.g., a knockout drum) for vapor-liquid phase separation. As illustrated, the cooled synthesis product stream 260 may be separated into a gas stream 270 and a liquid stream 275 in the feed separator 265. The liquid stream 275 may be introduced into a lower section of an HBr fractionator 280. In some embodiments, the HBr fractionator 280 may include a liquid distributor or manifold (not shown) to more evenly distribute the liquid stream 275 throughout the internal cross sectional area of the HBr fractionator 280. The HBr fractionator 280 may comprise a number of trays or equivalent packing material, identified in FIG. 5 by reference number 285. The gas stream 270 from the feed separator 265 may be further cooled, for example, to a temperature of about 10° C. to about 37° C., by exchanging heat in feed/overheads cross heat exchanger 290 with the HBr/hydrocarbon stream 70 from the overhead of the HBr fractionator 280 before the cooled gas stream 295 is introduced into a higher section of the HBr fractionator 280.

In accordance with present embodiments, the HBr fractionator 280 should separate CH₃Br and heavier hydrocarbons from the synthesis product stream 55 as a bottoms liquid product. As illustrated, the bottoms liquid product can be withdrawn from at or near the bottom of the HBr fractionator 280 via liquid CH₃Br/HC stream 295. The liquid CH₃Br/HC stream 295 should generally comprise CH₃Br and heavier hydrocarbons, such as C5+ hydrocarbons. Heavier alkyl bromides may also be present in the liquid CH₃Br/HC stream 295. In some embodiments, the liquid CH₃Br/HC stream 295 may be essentially free of HBr, for example, containing less than about 10 mppm and, alternatively, less than about 1 mppm. A second bottoms stream 300 comprising CH₃Br and other heavier hydrocarbons be withdrawn from at or near the bottom of the HBr fractionator 280 and vaporized in reboiler 305, for example, by means of steam 310 in a manner that will be evident to those of ordinary skill in the art before being introduced back into the HBr fractionator 280 at or near the bottom thereof. In some embodiments, the reboiler 305 may operate to heat the second bottoms stream 300 to a temperature of about 100° C. to about 230° C., and about 100° C. to about 200° C., in another embodiment.

An overhead vapor stream 315 may be withdrawn at or near the top of the HBr fractionator 280 and partially condensed in a reflux condenser 320 against a refrigerant 325 and conveyed to a reflux separator 330 (e.g., a separator drum). The reflux condenser 320 may operate to cool the overhead vapor stream 315 to a temperature of about −40° C. to about 0° C. In some embodiments, the overhead vapor stream 315 is cooled to a temperature warmer than about −40° C. and warmer than −34° C., in another embodiment. The reflux condenser 320 may have an operating pressure, for example, of about 5 barg to about 40 barg. The refrigerant 325 in the reflux condenser 320 may include, for example, propane or other available refrigerants. In the reflux separator 330, the overhead vapor stream 315 that was partially condensed in the reflux condenser 320 can be separated into a reflux stream 335 and the HBr/hydrocarbon stream 70. The reflux stream 335 may be conveyed via reflux pump 340 back into the HBr fractionator 280 at or near the top thereof. As illustrated, the HBr/hydrocarbon stream 70 exiting the reflux separator 330 may cross exchange in an overheads cross heat exchanger 345 with the overhead vapor stream 315 from the HBr fractionator 280 and in the feed/overheads cross heat exchanger 290 with the gas stream 270 from the feed separator 265. The HBr/hydrocarbon stream 70 from the reflux separator 330 may comprise, for example, HBr, methane (e.g., produced in the synthesis reactor 10 and/or residual methane), and C2-C4 hydrocarbons. In some embodiments, the HBr/hydrocarbon stream 70 may be essentially free of CH₃Br, for example, containing less than about 10 mppm CH₃Br and, alternatively, less than about 1 mppm. In accordance with present embodiments, the HBr/hydrocarbon stream 70 may be routed to other process units (e.g., the HBr separator 20 illustrated on FIGS. 1-4).

As illustrated, the liquid CH₃Br/HC stream 295 from the bottom of the HBr fractionator 280 may be routed to the CH₃Br recovery tower 350. Prior to entering the CH₃Br recovery tower 350, the liquid CH₃Br/HC stream 295 may be let down to a lower pressure, as desired for a particular application. In the illustrated embodiment, the liquid CH₃Br/HC stream 295 may be let down to a lower pressure across valve 355. The CH₃Br recovery tower 350 may operate, for example, at a pressure of about 1 barg to about 20 barg, and alternatively, about 3 barg to about 10 barg. In some embodiments, the CH₃Br recovery tower 350 may include a liquid distributor or manifold (not shown) to more evenly distribute the liquid CH₃Br/HC stream 295 throughout the internal cross sectional area of the CH₃Br recovery tower 350. The CH₃Br recovery tower 350 may comprise a number of trays or equivalent packing material, identified in FIG. 5 by reference number 360.

In accordance with present embodiments, the CH₃Br recovery tower 350 should separate the liquid CH₃Br/HC stream 295 into a CH₃Br stream 65 comprising CH₃Br and a C5+ stream 60 comprising pentane and heavier hydrocarbons. The C5+ stream 60 may also contain alkyl bromides heavier than CH₃Br, which can also be a bottoms product of the HBr fractionator 280, in some embodiments. The CH₃Br stream 65, for example, may also contain a quantity of C3-C4 hydrocarbons, which can also be a bottoms product of the HBr fractionator 280. As illustrated, the C5+ stream 60 can be withdrawn from at or near the bottom of the CH₃Br recovery tower 350. In some embodiments, the C5+ stream 60 may comprise less than about 10 mppm CH₃Br and, alternatively, less than about 1 mppm. In some embodiments, the C5+ stream may be essentially free of CH₃Br and HBr. As previously mentioned, the C5+ stream may be routed to the other process units (e.g., the dehydration and product recovery unit 25 as shown on FIG. 1). A second bottoms stream 365 comprising pentane and heavier hydrocarbons can be withdrawn from at or near the bottom of the CH₃Br recovery tower 350 and vaporized in reboiler 370, for example, by means of steam 375 in a manner that will be evident to those of ordinary skill in the art before being introduced back into the CH₃Br recovery tower 350 at or near the bottom thereof. In some embodiments, the reboiler 370 may operate to heat the second bottoms stream 365 to a temperature of about 150° C. to about 250° C. and about 150° C. to about 220° C., in another embodiment. It should be understood that the temperature of the reboiler 370 can be controlled, for example, to minimize the risk of the C5+ hydrocarbons degrading and resultant fouling.

The overhead vapor stream 380 may be withdrawn at or near the top of the CH₃Br recovery tower 350 and condensed in reflux condenser 385 against a coolant 390 and conveyed to a reflux drum 395. The reflux condenser 385 may operate to cool the overhead vapor stream 380 to a temperature warmer than about 37° C., in one embodiment, and warmer than about 43° C. in another embodiment. The coolant 390 in the reflux condenser 385 may include, for example, water, air, or other available cooling medium. The overhead vapor stream 380 that was condensed in the reflux condenser 142 can be fed to the reflux drum 395 from which an overhead condensate stream 400 can be withdrawn and fed to reflux pump 404. A portion of the overhead condensate stream 400 may be fed back into the CH₃Br recovery tower 350 at or near the top thereof as reflux stream 405. Another portion of the overhead condensate stream 400 may be routed to other process units as CH₃Br stream 65. The CH₃Br stream 65 may comprise, for example, residual CH₃Br and some hydrocarbons, such as C3-C4 hydrocarbons. In some embodiments, the CH₃Br stream 65 may comprise less than about 100 mppm C5+ hydrocarbons and, alternatively, less than about 10 mppm. In accordance with present embodiments, the CH₃Br stream 65 may be routed to the synthesis reactor 10 (e.g., FIGS. 1-2), shift reactor 210 (e.g., FIG. 3), light ends bromination reactor 235 (e.g., FIG. 4), or other suitable process unit depending, for example, on the particular application.

While the preceding description is directed to bromine-based processes for the conversion of lower molecular weight alkanes to higher molecular weight hydrocarbons, it should be understood that chlorine or another suitable halogen may be used in accordance with present embodiments. Additionally, it should be understood that embodiments of the present invention also encompass conversion of lower molecular weight alkanes to other higher molecular weight hydrocarbons. For example, a catalyst may be selected in the synthesis reactor 10 (e.g., shown on FIG. 1) for the production of olefins from alkyl bromides in a manner that will be evident to those of ordinary skill in the art.

To facilitate a better understanding of the present invention, the following examples of certain aspects of some embodiments are given. The following examples should not be read or construed in any manner to limit, or define, the entire scope of the invention.

### Example 1

Simulations were conducted to analyze the use of a debromination system for the recovery of CH₃Br in a process for converting natural gas to liquid hydrocarbons via a bromine-based method. The simulation was performed using a debromination system similar to the system illustrated by FIG. 5. A synthesis product stream comprising 0.79 mol % HBr, 0.04 mol % unconverted CH₃Br, 0.11 mol % C1-C4 alkanes, and 0.06 mol % C5+ hydrocarbons was cooled to a temperature of 60° C. and fed to an HBr fractionator at a rate of 2,995 kilogram moles per hour (kgmol/h). The HBr fractionator separated the feed into a 2,619 kgmol/h HBr/hydrocarbon stream (overhead) at 7.5 barg containing essentially all of the HBr and C1-C3 alkanes and a 376 kgmol/h liquid CH₃Br/HC stream (bottoms) containing CH₃Br and heavier hydrocarbons. The specifications of the HBr fractionator were 1 mppm HBr in the bottoms and 1 mppm CH₃Br in the overhead. The condenser temperature was −12.7° C. requiring a refrigeration duty of 3.9 MW. The reboiler temperature was 107° C. requiring a steam duty of 3 MW.

The HBr/hydrocarbon stream (overhead) from the HBr fractionator was fed to a CH₃Br recovery tower. The CH₃Br recovery tower separated the HBr/hydrocarbon stream into a 198 kgmol/h CH₃Br stream (overhead) containing essentially all of the CH₃Br at 3.6 barg and a 178 kgmol/h C5+ stream (bottoms) containing C5+ hydrocarbons. The specifications of the CH₃Br recovery tower were 1 mppm CH₃Br in the bottoms and 10 mppm C5+ in the overhead. The condenser temperature was 45° C. requiring a cooling duty of 5.4 MW. The reboiler temperature was 200° C. requiring a steam duty of 6.2 MW.

The above results are summarized in Table 1.

TABLE 1

| | | HBr | CH₃Br |
|---|---|---|---|
| | | Fractionator | Recovery Tower |
| Feed Rate | (kgmol/h) | 2,995 | 376 |
| Overhead Rate | (kgmol/h) | 2,619 | 198 |
| Bottoms Rate | (kgmol/h) | 376 | 178 |
| Condenser Temperature | (° C.) | −12.7 | 45 |
| Condenser Duty | (MW) | 3.9 | 5.4 |
| Reboiler Temperature | (° C.) | 107 | 200 |
| Reboiler Duty | (MW) | 3 | 6.2 |

Certain embodiments of the methods of the invention are described herein. Although major aspects of what is to believed to be the primary chemical reactions involved in the methods are discussed in detail as it is believed that they occur, it should be understood that side reactions may take place. One should not assume that the failure to discuss any particular side reaction herein means that that reaction does not occur. Conversely, those that are discussed should not be considered exhaustive or limiting. Additionally, although figures are provided that schematically show certain aspects of the methods of the present invention, these figures should not be viewed as limiting on any particular method of the invention.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Although individual embodiments are discussed, the invention covers all combinations of all those embodiments. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. All numbers and ranges disclosed above may vary by some amount. Whenever a numerical range with a lower limit and an upper limit is disclosed, any number and any included range falling within the range are specifically disclosed.

What is claimed is:

1. A process comprising:
reacting at least gaseous alkanes and a halogen to produce at least a halogenation product stream, wherein the halogenation product stream comprises alkyl halides, hydrogen halide, and unreacted alkanes;
reacting at least a portion of the alkyl halides from the halogenation product stream in the presence a catalyst to produce at least a synthesis product stream, wherein the synthesis product stream comprises unreacted methyl halide, higher molecular weight hydrocarbons, and hydrogen halide; and
separating the synthesis product stream into at least a first stream comprising hydrocarbons having five or more carbons, a second stream comprising unreacted methyl halide, and a third stream comprising hydrogen halide and hydrocarbons having one to four carbons.

2. The process of claim 1 wherein the halogen comprises bromine.

3. The process of claim 1 wherein the first stream comprises methyl halide in an amount of less than about 1 mppm, wherein the second stream comprises hydrocarbons having five or more carbons in an amount of less than about 10 mppm, and wherein the third stream comprises methyl halide in an amount of less than about 1 mppm.

4. The process of claim 1 wherein the step of separating the synthesis product stream comprises:
feeding the synthesis product stream into a first fractionator, wherein the third stream and a liquid stream are withdrawn from the first fractionator; and
feeding the liquid stream into a second fractionator, wherein the first stream and the second stream are withdrawn from the second fractionator.

5. The process of claim 4 wherein the step of separating the synthesis product stream further comprises one or more of the following steps:
cooling the synthesis product stream;
separating the synthesis product stream into a liquid fractionator feed stream and a gaseous fractionator feed stream; or
feeding the liquid fractionator feed stream and the gaseous fractionator feed stream into the first fractionator.

6. The process of claim 5 further comprising cooling the gaseous fractionator feed stream against the third stream.

7. The process of claim 4 wherein the first fractionator operates at a pressure of about 5 barg to about 40 barg.

8. The process of claim 4 further comprising:
withdrawing a second liquid stream from the first fractionator and heating the second liquid stream to a temperature of about 100° C. to about 230° C. in a reboiler;
withdrawing an overhead vapor stream from the first fractionator and cooling the overhead vapor stream to a temperature warmer than about −40° C. in a condenser; and
separating the cooled overhead vapor stream into at least the third stream and a reflux stream for feed into the first fractionator.

9. The process of claim 8 further comprising cooling the overhead vapor stream against the third stream.

10. The process of claim 4 further comprising reducing the liquid stream from the first fractionator to a pressure of about 1 barg to about 30 barg.

11. The process of claim 4 further comprising:
withdrawing a liquid stream from the second fractionator and heating the liquid stream to a temperature of about 150° C. to about 250° C. in a reboiler;
withdrawing an overhead vapor stream from the second fractionator and cooling the overhead vapor stream to a temperature warmer than about 37° C. in a condenser; and
separating the cooled overhead vapor stream into at least the second stream and a reflux stream for feed into the second fractionator.

12. The process of claim 1 further comprising recovering at least a portion of the hydrogen halide from the third stream.

13. The process of claim 1 further comprising:
separating the halogenation product stream into at least a gaseous stream and a liquid alkyl halides stream, wherein the gaseous stream comprises hydrogen halide and unreacted alkanes, and wherein the liquid alkyl halides stream comprises alkyl halides; and
separating the liquid alkyl halides stream into at least a monohalides stream and a polyhalides stream, wherein the monohalides stream comprises monohalogenated alkanes, and wherein the polyhalides stream comprises polyhalogenated alkanes,
wherein reacting at least a portion of the alkyl halides from the halogenation product stream comprises reacting at least a portion of the monohalogenated alkanes from the monohalides stream in the presence of the catalyst.

14. The process of claim 13 further comprising recovering at least a portion of the hydrogen halide from the gaseous stream.

15. The process of claim 13 further comprising reacting the gaseous alkanes with at least a portion of the polyhalogenated alkanes from the polyhalides stream to convert at least a portion of the polyhalogenated alkanes to monohalogenated alkanes.

16. The process of claim 13 further comprising recovering light end hydrocarbons from at least the third stream, the recovered light end hydrocarbons having from two carbons to four carbons.

17. The process of claim 16 further comprising feeding the polyhalides stream, the second stream, and the recovered light end hydrocarbons into a shift reactor to convert at least a portion of the polyhalogenated alkanes from the polyhalides stream to monohalogenated alkanes.

18. The process of claim 16 further comprising feeding the second stream, the recovered light end hydrocarbons, and a halogen into a light ends halogenation reactor to form a stream comprising alkyl halides and hydrogen halide, and reacting at least a portion of the alkyl halides from the stream in the presence of the catalyst.

19. The process of claim 1 wherein the catalyst comprises a synthetic crystalline alumino-silicate catalyst.

20. A process comprising:
reacting at least gaseous alkanes and bromine in a bromination reactor to produce at least a bromination product stream, wherein the bromination product stream comprises alkyl bromides, hydrogen bromide, and unreacted alkanes;
separating the bromination product stream into at least a gaseous stream and a liquid alkyl bromides stream, wherein the gaseous stream comprises hydrogen bromide and unreacted alkanes, and wherein the liquid alkyl bromides stream comprises alkyl bromides;
separating the liquid alkyl bromides stream into at least a monobromides stream and a polybromides stream, wherein the monobromides stream comprises monobrominated alkanes, and wherein the polybromides stream comprises polybrominated alkanes;
reacting at least a portion of the monobrominated alkanes from the monobromides stream in a synthesis reactor in the presence of a catalyst to produce at least a synthesis product stream, wherein the synthesis product stream comprises unreacted methyl bromide, higher molecular weight hydrocarbons, and hydrogen bromide; and
separating the synthesis product stream into at least a first stream comprising hydrocarbons having five or more carbons, a second stream comprising unreacted methyl bromide, and a third stream comprising hydrogen bromide and hydrocarbons having one to four carbons.

21. The process of claim 20 further comprising:
recovering at least a portion of the hydrogen bromide from the third stream in a hydrogen bromide separator;
providing a natural gas stream;
separating at least the third stream and the natural gas stream into at least a light ends product stream, a heavy ends product stream, and a feed gas stream, wherein the light ends product stream comprises light end hydrocarbons having from two carbons to four carbons, wherein the heavy ends product stream comprises heavy end hydrocarbons having five or more carbons, and wherein the feed gas stream comprises methane;
compressing the feed gas stream in a feed compressor;
feeding the feed gas stream into the bromination reactor;
generating a recycle alkane stream by recovering at least a portion of the hydrogen bromide from the gaseous stream in a hydrogen bromide separator;
compressing the recycle alkane stream in a recycle compressor; and
feeding the recycle alkane stream to the bromination reactor.

22. The process of claim 20 wherein the step of separating the synthesis product stream comprises:
feeding the synthesis product stream into a first fractionator, wherein the third stream and a liquid stream are withdrawn from the first fractionator; and
feeding the liquid stream into a second fractionator, wherein the first stream and the second stream are withdrawn from the second fractionator.

23. The process of claim 22 wherein the step of separating the synthesis product stream further comprises one or more of the following steps:
cooling the synthesis product stream;
separating the synthesis product stream into a liquid fractionator feed stream and a gaseous fractionator feed stream; or
feeding the liquid fractionator feed stream and the gaseous fractionator feed stream into the first fractionator.

24. The process of claim 23 further comprising cooling the gaseous fractionator feed stream against the third stream.

25. The process of claim 20 further comprising feeding the polybromides stream into the bromination reactor for reaction of at least a portion of the polybrominated alkanes in the polybromides stream with the gaseous alkanes to convert at least a portion of the polybrominated alkanes to monobrominated alkanes.

26. The process of claim 20 further comprising recovering light end hydrocarbons from at least the third stream, the recovered light end hydrocarbons having from two carbons to four carbons.

27. The process of claim 26 further comprising feeding the polybromides stream, the second stream, and the recovered light end hydrocarbons into a shift reactor to convert at least a portion of the polybrominated alkanes from the polybromides stream to monobrominated alkanes.

28. The process of claim 26 further comprising feeding the second stream, the recovered light end hydrocarbons, and a halogen into a light ends bromination reactor to form a stream comprising alkyl halides and hydrogen bromide, and reacting at least a portion of the alkyl halides from the stream in the synthesis reactor in the presence of the catalyst.

29. The process of claim 20 wherein the catalyst comprises a synthetic crystalline alumino-silicate catalyst.

30. A system comprising
a halogenation reactor configured for reaction of at least gaseous alkanes and a halogen to produce at least a halogenation product stream, wherein the halogenation product stream comprises alkyl halides, hydrogen halide, and unreacted alkanes;
a synthesis reactor in fluid communication with the halogenation reactor configured for reaction of at least a portion of the alkyl halides from the halogenation product stream in the presence of a catalyst to produce a synthesis product stream, wherein the synthesis product stream comprises unreacted methyl halide, higher molecular weight hydrocarbons, and hydrogen halide; and
a dehalogenation system in fluid communication with the synthesis reactor configured for separation of the synthesis product stream into at least a first stream comprising hydrocarbons having five or more carbons, a second stream comprising unreacted methyl halide, and a third stream comprising hydrogen halide and hydrocarbons having one to four carbons.

31. The system of claim 30, wherein the system further comprises:
an alkyl halides fractionation unit in fluid communication with the halogenation reactor configured for separation of the halogenation product stream into at least a gaseous stream and a liquid alkyl halides stream, wherein the gaseous stream comprises hydrogen halide and unreacted alkanes, and wherein the liquid alkyl halides stream comprises alkyl halides; and
a polyhalides fractionation unit in fluid communication with the alkyl halides fractionation unit configured for separation of the liquid alkyl halides stream into at least a polyhalides stream and a monohalides stream, wherein the polyhalides stream comprises polyhalogenated alkanes, and wherein the monohalides stream comprises monohalogenated alkanes;
